(19) United States
(12) Patent Application Publication
(10) Pub. No.: US 2004/0191790 A1
(43) Pub. Date: Sep. 30, 2004

(54) Novel regulatory protein

(76) Inventors: Monica Magdalena Maria Tomassen, Zeddam (NL); Hendrikus Christophel Petrus Maria Van Der Valk, Wageningen (NL)

Correspondence Address: YOUNG & THOMPSON, 745 SOUTH 23RD STREET 2ND FLOOR, ARLINGTON, VA, 22202

(21) Appl. No.: 10/476,667

(22) PCT Filed: Apr. 24, 2002
(86) PCT No.: PCT/NL02/00271

(30) Foreign Application Priority Data
May 4, 2001 (EP) 01201636.6

(51) Int. Cl. C12Q 1/68; C07H 21/04; C12N 9/24

(52) U.S. Cl. 435/6; 435/69.1; 435/320.1; 435/200; 435/325; 536/23.2

(57) ABSTRACT

Pectin molecules are mainly present in the middle lamella primary cell wall of young plant cells. The present invention pertains now to a protein involved in pectin modification. Furthermore, the invention also pertains to nucleic acid molecules encoding said protein. The protein can be used in inter alia methods of decreasing or increasing pectin degradation.

### FIELD OF THE INVENTION

The present invention is in the field of pectin modification and heat stabilisation. The invention is particularly concerned with a novel protein, derived from tomato, involved in pectin modification. Furthermore, the present invention relates to a method of producing the protein, to a method of producing an antibody specifically binding to said protein and to a method of decreasing or increasing pectin degradation in a preparation of interest.

### BACKGROUND OF THE INVENTION

In recent years the cultivated tomato, also called Lycopersicon esculentum, has become a popular system for studying fruit ripening. Ripening, the final phase of fruit development, is characterised by a series of co-ordinated biochemical and physiological changes which involves a number of dramatic metabolic changes in fruit tissues. An important aspect of the ripening process is fruit softening, which is thought to result primarily from modifications of the cell-wall. One of these modifications is the depolymerization and solublization of cell wall polyuronides by the ripening-induced cell wall degrading enzyme, polygalacturonase (PG). There is a correlation between the appearance of PG and the rate of softening and it is well-known that PG-activity increases dramatically during fruit ripening.

PG-activity in ripe tomato fruit is beleived to be due to the presence of four isoforms of PG. These four isoforms are structurally and immunologically related and prove to be, in part, the product of a single copy-gene that is developmentally regulated. The four isoforms are termed PGx, PG1, PG2a and PG2b. PG2a and PG2b differ only in the degree of glycosylation and have a molecular mass of 45 and 46 kDa, respectively. Because of the physical and biochemical similarity of PG2a and PG2b, these two isoforms can be treated as a single isoform, termed PG2. Transgenic anti-sense PG2 fruits show normal softening. This implies that softening is not only due to PG gene expression (Della Penna, 1987).

The heat unstable PG2 gene product can be transformed into the other PG isoforms with specific activities through binding with the so-called convertor (CV) (Knegt, 1988; Tucker, 1982). This CV is a protein with a molecular mass ranging from 81 kDa (Knegt, 1992) to 199.5 kDa (Moshrefi, 1983) and has no known enzyme activity of itself. It is assumed that CV has two bindingsites that can bind PG2. The binding of CV with one PG2 molecule results in PGx. PGx is a heat stable protein with a molecular mass of approximately 71 kDa, supposed to occur in situ in the fruit. When fruit tissue is homogenised CV can bind to two PG2 molecules and PG1 can arise. PG1 is a heat stable heterodimeric protein with a molecular mass of approximately 100 kDa, but this protein is believed to be an artefact occuring during homogenisation. On the other hand CV is never established as a single regulatory protein with pectin modification properties and no further molecular characteristics of CV have been described.

Other authors suggested that PG1 is derived from one PG2 molecule tightly associated with another cell wall glycoprotein, the so-called β-subunit (Zeng, 1992; Moore, 1994). The β-subunit is an acidic, heat stable, heavily glycosylated protein with an apparent molecular mass of 38 kDa (Pogson, 1991). The levels of β-subunit increase approximately 4-fold during ripening (Giovannoni, 1989; Pressey, 1984). However, anti-sense β-subunit plants demonstrate an increased release of pectic polysaccharides, indicating a PG inhibiting function of the β-subunit at most (Chun, 1997). Based on the above it can be concluded that the regulation of pectin modification, particularly the proteins involved, is still unclear, especially at the molecular level. A goal of the invention is to elucidate proteins binding and/or interacting to proteins involved in pectin modification such as polygalacturonases and thereby identifying regulatory mechanisms of pectin modification.

### SUMMARY OF THE INVENTION

A regulatory protein, also called activator, has now been isolated and purified from tomato and a part of its amino acid sequence and nucleotide sequence have been determined. The protein is involved in activation and stabilisation of pectin modifying enzymes. The present invention pertains to said protein, to nucleic acid molecules comprising nucleotide sequences encoding it, to cells containing nucleic acid constructs comprising said nucleic acid molecules, to methods of producing said protein and to compositions comprising said protein. The invention also pertains to an antibody specifically binding to said protein and to methods of decreasing or increasing pectin degradation, especially in fruit juices.

### DESCRIPTION OF THE INVENTION

The present invention covers a protein involved in pectin modification. More particularly, the invention relates to a protein having the ability to activate and heat stabilise the poygalacturonase activity of purified PG2, whereby the protein has a molecular weight of 8 kDa+2, preferably 1.5, more preferably 1 and particularly 0.5 kDa as determined by gelfiltration, and whereby the protein comprises a first amino acid sequence having at least 50%, preferably at least 55%, 60% or 65%, more preferably at least 70%, 75% or 80%, and particularly at least 85%, 90% or 95% amino acid identity as determined with the BLAST algorithm with the amino acid sequence of SEQ ID NO. 1.

Sequence identity is herein defmed as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1):387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, MD 20894; Altschul, S., et al., J. Mol. Biol. 215:40-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, WI. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gin; Ile to leu or val; Leu to ile or val; Lys to arg; gln or glu; Met to leu or ile; Phe to met, leu or tyr; Ser to thr; Thr to ser; Trp to tyr; Tyr to tip or phe; and, Val to ile or leu.

In a preferred embodiment of the invention the protein comprises a first amino acid sequence having the amino acid sequence of SEQ ID NO. 1.

As mentioned above the protein according to the invention has a molecular weight of 8±2 kDa as determined by gelfiltration on a Sephacryl S300 column using the molecular mass reference standard proteins blue dextran 2000, bovine serum albumin with a molecular mass of 67 kDa, trypsine with a molecular mass of 24 kDa and cytochrome C with a molecular mass of 12.5 kDa (see FIG. 1 and line 22 on page 12—line 10 on page 13 and lines 23-30 on page 18 of the examples section). SDS-PAGE analysis of reduced purified protein according to the invention confirmed this molecular weight determined by gelfiltration. In the presence of the protein according to the invention the activity of purified PG2 , determined by following pectin depolymerisation by measuring the reducing groups of pectin, i.e. a substrate for PG2 , using galacturonic acid as a standard, was shown to increase dramatically when compared to the activity in the absence of the protein (see FIG. 2 and lines 13-33 on page 13 and lines 1-11 on page 19 of the examples section). Next to that, the presence of the protein according to the invention has the ability to heat stabilise purified PG2 at temperatures of 65° C.

The invention further pertains to a protein according to the invention, whereby the protein further comprises a second amino acid sequence having at least 50%, preferably at least 55%, 60% or 65%, more preferably at least 70%, 75% or 80%, and particularly at least 85%, 90% or 95% amino acid identity as determined with the BLAST algorithm with the amino acid identity as determined with the BLAST algorithm with the amino acid sequence of SEQ ID NO. 12. The second amino acid sequence is the amino acid sequence of an internal peptide (Ala-Ala-Gly-Ile-Pro-Ser-Ala-Xaa-Gly-Val-Ser-Ile-Pro; SEQ ID No. 12) and this amino acid sequence is preferably located downstream of the first amino acid sequence with respect to the N-terminus of the protein.

The invention also relates to a protein according to the invention, whereby the protein comprises the amino acid sequence of SEQ ID No. 15. This amino acid sequence is the N-terminus of the purified protein, Leu-Ser-Cys-Gly-Gln-Val-Glu-Ser-Glu-Leu-Ala-Pro-Cys.

It was further determined by UV-spectrometry that the protein has an absorption maximum of about 211 nm. Chromatofocusing of the purified protein indicated that the iso-electric point (pI) of the protein is approximately 9.3. The activity of the protein can be measured in several parts of a plant, for instance in the root, the stem, the leaf and the fruit of a tomato plant. Furthermore, it is suggested that the protein according to the invention is a glycoprotein, particularly a lectin. Three myristoylation sites have been identified, GQVETG (SEQ ID No. 7; amino acid position 2-7 of SEQ ID No. 1), GLAPCL (SEQ ID No. 8; amino acid position 7-12 of SEQ ID No. 1) and GCCRGV (SEQ ID No. 9; amino acid position 23-28 of SEQ ID No. 1) suggesting that the protein may be covalently bound to the inner leaflet of the plasma membrane. Finally, it was shown that polyclonal antibody against purified β-subunit does not cross-react with the protein according to the invention. Furthermore, the amino acid sequence of the β-subunit has no homology with the amino acid sequence of the protein according to the invention, suggesting that both proteins are different.

A nucleic acid molecule comprising a nucleotide sequence encoding any of the above mentioned proteins, mutants or variants thereof is also a subject of the invention. In a particular embodiment of the invention the nucleic acid molecule according to the invention comprises a nucleotide sequence with a nucleotide sequence identity of at least 60%, preferably at least 65%, 70%, 75%, 80%, 85%, 90%, 95% as determined with the BLAST algorithm with the nucleotide sequence of SEQ ID No. 2. In a preferred embodiment of the invention the nucleic acid molecule according to the invention comprises the nucleotide sequence of SEQ ID No. 2. The above-mentioned nucleic acid molecules can be present in the genomic form, i.e. including the introns, or in the form which corresponds to the cDNA.

The invention is also concerned with the synthetic production of nucleic acid molecules comprising a nucleotide sequence encoding a protein according to the invention. Synthetic chemistry or recombinant technology may be used to introduce mutations such as deletions, insertions or substitutions into a nucleotide sequence encoding a protein according to the invention.

Nucleotide sequences corresponding to expression-regulating regions (for instance promoters, enhancers or terminators) located upstream or downstream a nucleotide sequence encoding a protein according to the invention are also a part of the invention. These expression-regulating regions can be used for homologous or heterologous expression of genes.

Furthermore, the invention also encompasses a nucleic acid molecule according to the invention comprising a nucleotide sequence operationally linked to one or more expression-regulating nucleotide sequences. The expression-regulating nucleotide sequences such as for instance a promoter, a ribosome binding site, a terminator, a translation initiation signal, a repressor gene or an activator gene can be any nucleotide sequence showing activity in the host cell of choice and can be derived from genes encoding proteins, which are either homologous or heterologous to the host cell of choice. The expression-regulating nucleotide sequences will largely depend on the vector and the host cell used.

Also encompassed by the present invention are nucleic acid molecules comprising nucleotide sequences that are capable of hybridizing to any of the above nucleotide sequences, in particular to the nucleotide sequence of SEQ ID No. 2, under various conditions of stringency such as for instance 0.1*SSC at a temperature of 42° C. or 0.5*SSC at a temperature of 50° C.

In another embodiment of the invention a nucleic acid molecule according to the invention may comprise a nucleotide sequence ligated to a heterologous nucleotide sequence to encode a fusion protein to facilitate protein purification and protein detection on for instance Western blot and in an ELISA. Suitable heterologous sequences include, but are not limited to, the nucleotide sequences encoding for proteins such as for instance glutathione-S-transferase, maltose binding protein, metal-binding polyhistidine, green fluorescent protein, luciferase and beta-galactosidase. The protein may also be coupled to non-peptide carriers, tags or labels that facilitate tracing of the protein, both in vivo and in vitro, and allow for the identification and quantification of binding of the protein to substrates. Such labels, tags or carriers are well-known in the art and include, but are not limited to, biotin, radioactive labels and fluorescent labels.

A nucleic acid construct comprising a nucleic acid molecule according to the invention is also part of the invention. In a specific embodiment of the invention, a nucleic acid construct according to the invention comprises a nucleic acid molecule according to the invention positioned in such a way that an antisense form of the RNA of a protein according to the invention is produced. In another particular embodiment of the invention, a nucleic acid construct according to the invention comprises a nucleic acid molecule comprising a nucleotide sequence encoding a protein according to the invention with a mutation leading to expression of a non-functional form of said protein or leading to complete absence of expression of said protein. Suitable nucleic acid constructs according to the invention include, but are not limited to, vectors, in particular plasmids, cosmids or phages. The choice of vector is dependent on the recombinant procedures followed and the host cell used. Vectors that can be used when a bacterium is utilized as a host cell include, but are not limited to, bacteriophage, plasmid, or cosmid DNA expression vectors. When yeast is used, yeast expression vectors are preferred, while in case of the utilization of insect cells, virus expression vectors such as for instance baculovirus are preferred. Plant cells are preferably transformed with virus expression vectors such as inter alia cauliflower mosaic virus or tobacco mosaic virus or bacterial expression vectors such as inter alia Ti or pBR322 plasmids. The vectors may be autonomously replicating vectors or may replicate together with the chromosome into which they have been integrated. Preferably, the vector contains a selection marker. Useful markers are dependent on the host cell of choice and are well known to persons skilled in the art.

A transformed host cell, such as a mammalian (with the exception of human), plant, animal, insect, fungal, yeast or bacterial cell, containing one or more copies of the nucleic acid constructs mentioned above is an additional subject of the invention. Examples of suitable bacteria are Gram positive bacteria such as several Bacillus or Streptomyces strains or Gram negative bacteria such as Escherichia coli. For optimal expression (secretion into the culture medium) and protein maturation (folding) in bacteria it may be necessary to insert a bacterial signal peptide coding sequence such as the presequence of an amylase gene from a Bacillus species and to introduce an alternative translational start codon. Expression in yeast can be achieved by using yeast strains such as Pichia pastoris, Saccharoniyces cerevisiae and Hansenula polymorpha. For optimal expression (secretion into the culture medium) and protein maturation (folding) it may be necessary to insert a yeast α-factor signal sequence and to introduce an alternative translational start codon. Alternatively, a suitable expression system can be a baculovirus system or expression systems using mammalian cells such as COS or CHO cells. In a preferred embodiment of the invention the host cells are plant cells, preferably cells of tomato. Transformed (transgenic) plants or plant cells are produced by known methods, for example, by transformation of leaf discs, by co-culture of regenerating plant protoplasts or cell cultures with Agrobacterium tumefaciens or by direct DNA transfection. Resulting transformed plants are identified either by selection for expression of a reporter gene, or by expression of the protein of interest. For optimal expression (secretion into the culture medium) and protein maturation (folding) it may be necessary to use a signal peptide coding sequence of a plant. In a specific embodiment of a transformed host according to the invention a nucleic acid construct comprising a nucleotide sequence according to the invention which is positioned so that an antisense form of the RNA of a protein according to the invention is produced. In this specific embodiment the host cell is preferably a plant cell and more preferably a tomato cell.

Additionally, the invention relates to a method of producing a protein according to the invention. This protein may be isolated, partially or completely, from the culture of a plant, such as a tomato plant, preferably a green tomato, naturally expressing said protein. In plants the protein may preferably be recovered from the pericarp but other parts of the plant expressing the protein can also be used. The specific isolation and purification procedures of the protein from tomato pericarp are illustrated hereafter in the Examples section. Alternatively, the protein may also be isolated from a transformed organism expressing said protein. In transformed host cells the protein can be recovered from the cell free extract or preferably from the culture medium by means known by persons skilled in the art. Furthermore, a protein according to the invention or a part thereof, may be synthesized using chemical methods known in the art.

Furthermore, the invention concerns a composition comprising a protein according to the invention. Said composition can further comprise compounds not affecting the functionality of the protein. These compounds can be standard compounds known in the art. Preferably, said composition comprises an extract of a transformed host according to the invention or an extract of the culture medium thereof. More preferably, said composition comprises an extract of a plant expressing a protein according to the invention.

Pectin degradation can lead to inter alia fruit softening. This fruit softening can be decreased by for instance decreasing pectin degradation. The invention now also pertains to a method to decreasing the degradation of pectin in cells, preferably fruit cells. Decreasing pectin degradation can be useful in for instance fresh fruit products, fruit processing, for instance tomato processing such as production of tomato-based puree or paste, the production of jam and jelly and fermentation procedures. In fresh fruit products the decrease in pectin degradation leads to crisp fruit with a better storage life. In for instance production of tomato-based puree and paste the decrease in the pectin degradation leads to purees and pastes that are less aqueous after hot break and will allow low processing temperatures (cold break). Furthermore, in the production of jam and jelly a decrease in pectin degradation leads to a more controlled production process. Pectin degradation can be decreased or even deleted, when the levels of (active) proteins involved in pectin degradation are decreased or deleted. These proteins include, but are not limited to, polygalacturonase and other pectin-modifying proteins. The present invention encompasses a method of decreasing pectin degradation comprising transforming a cell, preferably a fruit cell, expressing a protein according to the invention with a nucleic acid construct wherein a nucleotide sequence encoding a protein according to the invention is positioned so that an antisense form of the RNA of said protein is produced, such that said cell will comprise a lowered level of a functionally active pectin degrading protein such as for instance polygalacturonase isoform I and/or X, preferably a lowered level of a pectin degrading protein such as for instance polygalacturonase isoform I and/or X and even more preferably no pectin degrading protein such as for instance polygalacturonase isoform I and/or X after transformation. A transformed host, preferably a plant cell, comprising a nucleic acid construct, wherein a nucleic acid molecule comprising a nucleotide sequence encoding a protein according to the invention is positioned so that an antisense form of the RNA of said protein is produced, comprising a lowered level of a functionally active pectin degrading protein such as for instance polygalacturonase isoform I and/or X, preferably a lowered level of a pectin degrading protein such as for instance polygalacturonase isoform I and/or X and even more preferably no pectin degrading protein such as for instance polygalacturonase isoform I and/or X is also part of the invention.

In another embodiment of the invention a method of decreasing the degradation of pectin in fruit comprises transforming a fruit cell expressing a protein according to the invention comprising a nucleic acid construct comprising a nucleic acid molecule comprising a nucleotide encoding a protein according to the invention with a mutation leading to expression of a non-functional form of said protein or leading to complete absence of expression of said protein. A consequence of this could be the absence of a functionally active pectin degrading protein such as for instance a functionally active polygalacturonase isoform I and/or X, preferably a lowered level of a pectin degrading protein such as for instance a polygalacturonase isoform I and/or X and even more preferably no pectin degrading protein such as for instance polygalacturonase isoform I and/or X. Mutation and transformation techniques for performing the above method are known for a person skilled in the art. Other methods known in the art of decreasing the degradation of pectin in fruit by lowering or deleting the level of a functionally active form of a protein according to the present invention such as inter alia selection of low- or zero-activator cultivars in plant breeding are also part of the invention. A transformed host, preferably a plant cell, comprising a nucleic acid construct, wherein the nucleic acid construct comprises a nucleic acid molecule comprising a nucleotide sequence encoding a protein according to the invention comprising a mutation leading to expression of a non-functional form of said protein or leading to complete absence of expression of said protein is also part of the invention.

The invention further relates to a method decreasing or deleting the interaction between a protein according to the invention and a pectin degrading protein such as one of the polygalacturonase isoform II proteins, leading to a decrease in pectin degradation. The interaction can be decreased or even deleted in the cell or in any composition of interest comprising an active pectin degrading protein and a protein according to the present invention by manipulating physical binding conditions by inter alia modifying the salt concentration, the temperature and the pH in such a way that for instance polygalacturonase isoforms I and/or X are not formed.

The invention further encompasses a method of increasing pectin degradation by adding a protein according to the invention and/or a composition comprising said protein to a preparation of interest. A particular embodiment of the invention concerns a method of increasing pectin degradation in a cell comprising transforming said cell with a nucleic acid construct comprising a nucleic acid molecule comprising a nucleotide sequence encoding a protein according to the present invention and culturing said cell under conditions conductive to expression of the protein. Pectin and/or other pectic substances comprise approximately 0.5-4% of the weight of fresh (fruit) material. In for instance juice production it may be desirable to a add a protein according to the invention to increase the degradation of pectin and/or pectic substances, since these substances give rise to inter alia high viscosity of the fruit juice, low pressability of the pulp obtained when the fruit tissue is ground and development of a jelly structure comprising pulp and high viscosity juice. When a protein according to the invention is added during the production of juices, said juices are easily obtained and with higher yields. Particularly, in the case of sparkling clear juices which include, but are not limited to, apple juice, pear juice, grape juice, strawberry juice, raspberry juice, blackberry juice and wine, a protein according to the invention is added in order to increase the juice yield during processes such as pressing and straining of the juice and to remove suspended matter. In apple juices a protein according to the invention can be added to facilitate pressing or juice extraction and to help in the separation of a flocculent precipitate by sedimentation, filtration or centrifugation. A protein according to the invention allows a producer to diversify the type of products, i.e. cloudy, clear apple juices, clear apple concentrates, etc., and to increase the value of the raw material. Furthermore, the total time for juice extraction is shortened, the juices and concentrates become more stable and have an improved taste and the costs for production are reduced because of the higher yield and the use of less equipment and labor. In a particular form of apple juice, called cider, a protein according to the invention can be added to control and accelerate the mechanism of formation of a gel of pectinates, which can be removed from the juice. In pear juices a protein according to the invention can be added to facilitate juice processing by improving the cloud stability in nectars and the ability to concentrate the product and to decrease the viscosity of the juice. In grape juice and in wine a protein according to the invention can be added to facilitate flocculation of insoluble particles and to reduce haze or gelling of the juice at any of the stages involved in grape juice production. In strawberry juice, raspberry juice and blackberry juice a protein according to the invention can be added to facilitate the production of clear juices and concentrates from the fruits, since high amounts of pectin make the juices viscous and therefore difficult to clarify, filtrate and concentrate.

In the case of cloudy juices which include, but are not limited to, orange juice, lemon juice, mango juice, apricot juice, guava juice, papaya juice, pineapple juice and banana juice, a protein according to the invention is added in order to stabilize the cloud of citrus juices, purees and nectars. In orange juices a protein according to the invention can be added to reduce viscosity of the juice or to increase cloud stability. In lemon juices a protein according to the invention can be added to improve the recovery of essential (peel) oils by improving the process time, yield of oil from an water-oil emulsion obtained during the recovery of the oils and the quality of the final product. Furthermore, a protein according to the invention can be used in the preparation of citrus salads and dried animal feed. In mango juices a protein according to the invention can be added to increase the concentration of mango puree, to degrade mango pulp and to produce clear juices and concentrates. In apricot juices a protein according to the invention can be added to improve the cloud stability of nectar. In guava juices a protein according to the invention can be added to improve juice extraction, particularly clarification of the juices. In papaya juices a protein according to the invention can be added to improve concentration of the juices and in pineapple juices a protein according to the invention can be added to obtain clear concentrates. In banana juices a protein according to the invention can be added to reduce viscosity, give high yield and concentration of said juices.

Furthermore, a protein according to the invention can also be added to for instance processes generating unicellular products that can be used as base material for pulpy juices and nectars, as baby foods, as ingredients for the dairy products such as pudding and yogurts and as protoplasts for various biotechnological applications. Particular processes include, but are not limited to, maceration of plant tissue, liquefaction, saccharification and flavour extraction of biomass and isolation of protoplasts.

Besides that, a protein according to the invention can also be used to improve retting, i.e. a fermentation process wherein certain bacteria and fungi decompose pectin of the bark and release fiber and degumming of fiber crops. Furthermore, a protein according to the invention can be used in pretreatment of pectic wastewater from fruit juice industries improving the efficiency, lowering the costs and the complexity of the process and decreasing the treatment period. A protein according to the invention can also be used to improve the manufacturing process of Japanese paper and paper in general, to improve oil extraction and increase the stability of the extracted oil, such as olive oil, sunflower seed oil etc., and to improve the fermentation of coffee and tea and particularly the foam-forming properties of instant tea powders. A protein according to the invention can further be used to improve fiber modification, to improve processing of fruit and in technological applications such as in filters. In the processing of fruit an increase in pectin degradation gives rise to a lowered process temperature, a shorther production time and a better quality of the processed fruit. A protein according to the present invention can also be bound or otherwise adhered to a filter. This filter can for instance be used to bind pectin-modifying proteins, such as polygalacturonase, present in a preparation of interest.

The invention concerns antibodies which specifically bind to the protein according to the invention. As used herein, the tern antibodies includes inter alia polyclonal, monoclonal, chimeric and single chain antibodies, as well as fragments (Fab, Fv, Fa) and an Fab expression library. Such antibodies against a protein according to the invention can be obtained as described hereinbelow or in any other manner known per se, such as those described in WO 95/32734, WO 96/23882, WO 98/02456 and/or WO 98/41633. For instance, polyclonal antibodies can be obtained by immunizing a suitable host such as a goat, rabbit, sheep, rat, pig or mouse with a protein according to the invention or an immunogenic portion, fragment or fusion thereof, optionally with the use of an immunogenic carrier (such as bovine serum albumin or keyhole limpet hemo-cyanin) and/or an adjuvant such as Freund's, saponin, ISCOM's, aluminium hydroxide or a similar mineral gel, or keyhole limpet hemocyanin or a similar surface active substance. After an immune response against the protein according to the invention has been raised (usually within 1-7 days), the antibodies can be isolated from blood or serum taken from the immunized animal in a manner known per se, which optionally may involve a step of screening for an antibody with desired properties (i.e. specificity) using known immunoassay techniques, for which reference is against made to for instance WO 96/23882. Monoclonals may be produced using continuous cell lines in culture, including hybridoma and similar techniques, again essentially as described in the above cited references. Fab-fragments such as F(ab)₂, Fab′ and Fab fragments may be obtained by digestion of an antibody with pepsin or another protease, reducing disulfide-linkages and treatment with papain and a reducing agent, respectively. Fab-expression libraries may for instance be obtained by the method of (Huse et al., 1989). Preferably, a monoclonal antibody against the protein according to the invention is used, more specifically against the protein comprising the amino acid sequence shown in/encoded for by SEQ ID No 1 and/or SEQ ID No 12 and/or SEQ ID No 15 or (an antigenic) part thereof; and such monoclonals are a further aspect of the invention. In a further aspect, the invention provides a cell line such as a hybridoma that produces antibodies, preferably monoclonal antibodies, against a protein according to the invention, more specifically against the protein comprising the amino acid sequence shown in/encoded for by SEQ ID No 1 and/or SEQ ID No 12 and/or SEQ ID No 15 or (an antigenic) part thereof. The invention also relates to a method for producing an antibody, preferably a monoclonal antibody, against a protein according to the invention, more specifically against the protein comprising the amino acid sequence shown in (or encoded for) by SEQ ID No 1 and/or SEQ ID No 12 and/or SEQ ID No 15 or (an antigenic) part thereof, said method comprising cultivating a cell or a cell line that produces said antibody and harvesting/isolating the antibody from the cell culture.

### EXAMPLES

A novel regulatory protein (further also referred to as activator) has been isolated and purified from tomato. Several characteristics of said protein have been determined.

Activator Isolation and Purification

Mature green tomato pericarp was cut into 1.0-cm pieces, mixed with an equal volume of cold water in a small plastic bag (format 15*20 cm and 0.02 mm thick), followed by 5 min heating at 65° C. in a shaking water bath. After cooling down on melting ice for 20 min, the mixture was homogenised in a cooled Bülher for 1 min. The pH of the suspension was lowered to pH 3.0 with 1.0 M HCl. After that, the suspension was stirred for 15 min at room temperature and centrifuged at 20,000 rpm at 4 ° C. for 15 min. The pellet was homogenised in 1.25 M NaCl solution, twice the volume of the previous amount of water. The suspension was adjusted to pH 5.5 with 1.0 M NaOH, stirred for 15 min at room temperature and centrifuged for 15 min at 4° C. at 20,000 rpm. The supernatant was filtered through a 0.45 μm mesh filter and stored at −20° C. The supernatant was successively dialysed against 100 mM sodiumacetate (NaAc) buffer with 200 mM NaCl, pH 4.5 and concentrated using Amicon filtration system (YM10 filters) After that, gelfiltration was performed on a Sephacryl S300 column with a length of 1.0m and an internal diameter of 16 mm. The column was eluated with 100 mM NaAc buffer, pH 4.5 containing 200 mM NaCl with a linear flow rate of 8.4 ml/h and samples of 2-2.5 ml were applied. All samples were UV-measured by 280 nm. For determination of the molecular mass reference standards were used. These standards were blue dextran 2000; molecular mass is 2000 for void volume determination, bovine serum albumin, molecular mass is 67 kDa, Trypsine, molecular mass is 24 kDa and Cytochrome C, molecular mass is 12.5 kDa. The unknown molecular mass of the protein according to the invention was calculated from their relative retention volumes.

Activator Activity Assay In Vitro.

The activity of the activator was determined by following the existence of reducing sugars, which were formed by the depolymerization of pectin through PG2 and activator action. The reducing groups were measured with a modified ferricyanide method (Rozie, 1988; Robijt, 1973) using D-galacturonic acid as a standard. The reaction mixture contained an enzyme solution comprising purified PG2 and activator filled up to 250 μl with NaAc buffer containing 100 mM NaAc and 200 mM NaCl, pH 4.5. The reaction mixture was incubated for 40 min at 37° C. in a shaking water bath, followed by a PG inactivation at 65° C. for 5 min also in a shaking water bath. After cooling down the reaction mixture on ice for 20 min, 1 ml 0.25% Citrus pectin dissolved in NaAc buffer, pH 4.5 was added. The degree of esterification of the pectin-substrate was 63-66% and the degree of methylation was 6.7%. Directly after the pectin solution was added, a sample of 100 μl (T=0) was taken and stored on ice. The reaction mixture was placed back in the shaking water bath by 37° C. and every 15 min samples of 100 μl were taken and also stored on ice. When all samples were collected, 900 μl of a yellow coloured freshly prepared mixture of a cyanide solution (0.125% KCN, 0.04% Fe(CN)₆, 1% Na₂CO₃) was added. The samples were incubated at room temperature for 20 min, boiled for 10 min in a shaking water bath and immediately cooled on ice. After 1 h at room temperature the decolorization was measured spectrophotometrically at 420 nm. The activity of the enzyme was expressed as the number of moles of galacturonic acid units (GA-units), reducing groups produced per minute.

Activator Activity in Different Plant Parts

To investigate whether the protein is present in other parts of the tomato plant, the protein was isolated from fruit, leaves, roots and the stem. After isolation of the activator, pectin degradation was measured using the above mentioned method. A mixture of purified fruit PG2 with isolated root-, stem-, fruit- or leaf-activator was used as a reaction mixture. The activities of the PG2 -activator complex were calculated using calibration curves with galactuonic acid coupled with the slope of the activity assay. The activity was expressed in nmol/h/kg of the part of the plant used.

Activator Activity Assay In Situ (i.e. In Vivo).

The activity of the protein was measured in situ using tomato pericarp discs. The discs were prepared with a cork border (12 mm diameter) and fixed in a metal frame with a thichness of 2 mm. The internal epidermal tissues were removed with razor blades. The resulting discs (2 mm thick) were treated with 25 μl NaAc buffer (100 mM NaAc, 200 mM NaCl, pH 4.5) and NaAc with activator. Before treatment, the solutions were heated 5 min 65° C. Discs were incubated in a covered petri dish over moistened filter paper for 2 h at 37° C. and dried for a short period by turning the discs on tissue paper. The discs were processed for cryo scanning microscopy briefly, sections of tissue were fixed in a SEM-holder with a tissuetek, and frozen with melting nitrogen and sputter-coated with gold (JeolXX) all this take place under cryo circumstances. The surface of a disc was observed and SE images were made using a cryo Jeol scanning electron microscope 5600-LV at −193° C.

Activator Characterisation

The UV-spectrum of gelfiltrated, purified protein was measured with an UV spectrofotometer (Pharmacia) and the absorption maximum of the protein was determined.

The molecular structure of the purified protein was analysed by sodium dodecyl sulphate-polyacrylamide gel electrophoresis (SDS-PAGE) on a PhastSystem (Pharmacia) electrophoresis apparatus or with a Mini-protean II cell (BioRad). Proteins were separated on several polyacrylamide gels, for example precast commercial gels (PhastGel gradient 8-25%, PhastGel homogeneous 20% and High density gels) (Pharmacia), self made 7.5 up to 15% polyacrylamide gels and 15 up to 25% SDS tricine polyacrylamide gels (Laemmli, 1970). Precast gels were silver-stained with the PhastSystem development unit according to the suppliers' protocol. The self-made gels were stained with Coomassie Blue (supplier's protocol BioRad) or silver stained according to Morrisey (1981). Molecular weight markers run for comparison were Myoglobin III (2512 Da), Myoglobin II (6214 Da), Myoglobin 1 (8159 Da), Myoglobin I+II (14404 Da) and Myoglobin (16949 Da) or LMW marker from Pharmacia).

Determination of the iso-electric point (pI) of the purified protein was performed by iso-electric focussing on a PhastGel IEF 3-9 (Pharmacia) using a PhastSystem (Pharmacia) electrophoresis apparatus. Broad pI markers (pI 3.5-9.3 Pharmacia) were used to determine the profile of the pH gradient. Gels were stained with silver according to the manufacturer's instructions (Pharmacia).

To demonstrate that the activator is not identical to the β-subunit, polyclonal antibodies against the purified β-subunit were used. These antibodies were obtained from T. Moore (UC Davis, Calif.). In short, β-subunit was purified and resuspended in water, emulsified in Freund's complete adjuvant, and injected subcutaneously at multiple sites into a rabbit as described by Harlow and Lane (1988). Non-denaturing PAGE was performed at 4° C. on 7.5% polyacrylamide gels essentially as described by Moore et al. (1994). After electrophoresis, gels were washed in Tris transport buffer (Tris-base, Glycine, SDS and MeOH) three times for 10 min and the proteins were electroblotted on nitrocellulose (0.2 μM) during 45 min by 20 V. Next, the blots were incubated with 2% non-fat dry milk (nfdm, DMV Campina), 0.1% Tween 20 in 1 time Tris-buffered saline (TBS, 25 mM Tris-HCl, pH 7.6, 155 mM NaCl) for 1 h at 60° C. Then the blots were incubated for 1 h at room temperature in 15 ml 1 time TBS with 0.1% nfdm, 0.1% triton-x 100 and 15 μl anti-β-subunit antibody. After the blots were washed three times for 10 min with 1 time TBS with 0.1% Triton X-100, each of the blots were incubated for 1 h with alkaline phosphatases-conjugated goat anti-rabbit antibody (BioRad). The blots were washed again in 1 time TBS and developed with nitroblue tetrazolium chloride and 5-bromo-4-chloro-β-indolylphosphate according to the manufacturer's protocol (Boehringer Mannheim Biochemica).

Glycoprotein analysis was realised by a modified PAS-staining according to Kapitany et al. (1973). Proteins were spotted on a nitrocellulose membrane and incubated with 12.5% TCA for 1 hr. After that, the membrane was incubated with 1% periodic acid with 3% acetic acid and washed 6 times with 15% acetic acid during the time period of 1 hr. These steps were all carried out at room temperature. The proteins were coloured with Schiff s Reagent at 4° C. in the dark for 1 hr, followed by an incubation of 2 times 5 min with 0.5% sodium metabisulfite. The membranes were destained with 7% acetic acid.

Agglutination assays were performed on cavity slides. Therefore, rabbit erythrocyte cells were washed 3 times with physiological saline (0.9% NaCl) and resuspended in 12 times the original volume. Aliquots of 10 μl erythrocyte cells were mixed with different amounts of protein solutions filled up to an equal volume with physiological saline. The protein solution was obtained by ethanol-precipitation of purified activator with an end-concentration of 0.08 μg/μl protein. The slides were incubated for 10 min at room temperature and the scoring was done with the unaided eye.

Protein Sequencing and Amino Acid Analysis

Prior to protein sequencing or amino acid analysis the protein was, besides purified by gelfiltration, also purified by analytical HLPC. Analytical HPLC was performed on a Waters MSDS 600E HPLC system equipped with a Waters 990 photodiode array detector (Waters, Eschbom, Germany) using a Nucleosil 10 C18 (150×2 mm) column (Waters, Eschbom, Germany). The column was eluted with a 120 min linear gradient from 0 to 100% acetonitrile in 0.05% TFA. The N-terminal sequencing, internal peptide sequencing and amino acid analysis was carried out by Eurosequence BV Groningen. In short, the proteins were pyridylethylated and degraded using the Edman procedure as described by Edman (1956). After that, the sequences of the N-terminus and an internal peptide were determined on an automated sequenator (Model 477A, Applied Biosystems, Hewick et al., 1981) coupled with a HPLC (Model 120A ABI). The amino acid analysis were committed with a HP 1090 Aminoqant (Schuster, 1988) by means of a two-steps derivation with OPA and FMOC.

Nucleotide Sequencing

Green tomato fruits were collected from greenhouse grown tomato plants and frozen with liquid nitrogen. Total RNA was extracted via the following method: 50 mg of frozen tissue was ground to a powder in liquid nitrogen with a mortar and pestle. The ground powder was vortexed for 30 seconds with 0.75 ml extraction buffer (1% sarkosyl, 20 mM EDTA, 100 mM NaCl and 100 mM Tris-HCl, pH 8.5), followed by centrifugation for 5 min in an Eppendorf centrifuge (max. speed). The supernatant was extracted twice with phenol/chloroform (1:1) and once with chloroform. After centrifugation for 10 min, RNA was isopropanol-precipitated and the resulting pellet was washed with 70% ethanol. The ethanol was removed by evaporation and the pellet was dissolved in 750 μl DEPC-water. After that, 250 μl 8 M LiAc was added to the pellet and the mixture was incubated for 3 hours on ice. Thereafter, the mixture was centrifuged for 20 min and the pellet was dissolved in 0.4 ml DEPC-water. 0.04 ml 3 M NaAc and 1.0 ml 96% ethanol was added to the mixture and the mixture was incubated at −80 ° C. during the night. The mixture was centrifuged for 10 min and washed with 150 μl 80% ethanol and centrifuged again. After removing the supernatant, the pellet was dried on air, dissolved in 20-100 μl DEPC-water with 0.5 μl RNA-guard and stored at −80° C. From the obtained RNA cDNA was made using RT-PCR. Therefore, oligonucleotide primers corresponding to the amino acid sequences of the N-terminus and an internal peptide were used. The amino acid sequence of the N-terminus was: Leu-Ser-Cvs-Glv-Gln-Val-Glu-Ser-Glu-Leu-Ala-Pro-Cys (SEQ ID No. 15) and the amino acid sequence of the internal peptide was: Ala-Ala-Gly-Ile-Pro-Ser-Ala-Xxx-Gly-Val-Ser-Ile-Pro (SEQ ID No. 12). The underlined amino acids of the sequences indicate the sequence from which nucleotide primers were constructed by Pharmacia Biotech. The sequence from the forward oligonucleotide primer of the N-terminus was: GGAATTCCTG(T/C)GGNCA(G/A)GTNGA(GIA)(T/A)(C/G)NGG (SEQ ID No. 13) and the sequence of the reverse primer, affected from the internal peptide, was GGAATTCCGCN(G/C)(A/T)NGGNATNCCNGCNGC (SEQ ID No. 14). In both primers N indicates the presence of an inosine. Both primers started with an EcoR1 site. RT-PCR reactions were performed with 5 μg RNA and 100 pmol reverse primer filled up to 11 μl with RNase-free water, followed by heating during 5 min at 65° C. and cooling until 25° C. After that, 9 μl reverse transcription reaction mix, containing 2 μl 2, mM dNTP, 2 μl 0.1 M DTT, 4 μl 5 times reaction buffer (Boehringer) and 1 μl reverse transcriptase (GIBO M-MLV, 510-8025S) was added. The solution was incubated successively 10 min at 25° C., 45 min at 37° C. and 5 min at 95° C. After that, 80 μl polymerase chain reaction mix (PCR mix) containing 1 μl forward primer (100 pmol), 8 μl 2 mM dNTP, 8 μl 10 times reaction buffer (Boehringer), 0.5 μl Taq-polymerase Boehringer) and 62.5 μl water was added. PCR was performed with cDNA as template on a thermocycler (Perkin-Elmer) using 40 cycles with the following temperature profile: 30 sec at 95° C., 30 sec at 45° C. and 1 min at 72° C. After electrophoresis, fragments of the expected length were isolated using the Qiaex agarose gel extraction kit (Qiagen) according to the suppliers' protocol. The purified PCR-fragment was digested with EcoR1 and cloned into the pBluecript vector. The ligation mixture was heated at 65° C. for 10 min and dialysed for 1 h against 10% glycerol and electroporated into E.coli cells. These cells were plated and recombinant plasmids were screened for the correct insert using blue/white screening and/or size comparison to the original PCR fragment. E. coli cells comprising said PCR-fragment are deposited at the CBS. Brief description of this method: E.coli cells were grafted into 19 μl water, 0.5 l reverse M13 primer and 0.51 μl forward M13 primer. Followed by incubation at 96° C. for 8 min. After that, 2 μl 2 mM dNTP, 2.5 μl PCR mix and Mg and 2 μl Taq-polymerase was added and the mixture was incubated at 94° C. for 2 min. PCR was performed with E.coli DNA as template with the following temperature profile: 30 sec at 94° C., 1 min at 45° C., 1 min at 72° C. and a final extension of 10 min at 72° C. The presence of the recombinant plasmids was tested on an agarose gel. From the clones comprising recombinant plasmids, DNA was isolated and the positive clones were determinded using a T7 sequencing kit (Pharmacia Uppsala).

Results

Activator Protein Isolation and Purification

Green tomato fruit was selected as starting material for the isolation of the activator because it is known that tomatoes in the mature green stage do not possess PG-enzyme activity. The activator binding site should be free and available for PG2 . For that reason the pericarp tissue was heated for 5 min at 65° C., to be sure that no possible bound PG affects the activator. Gelfiltration on Sephacryl S300 was an effective step to isolate the activator (FIG. 1). The relative molecular mass of the activator was determined to be 8 kDa The molecular mass was calibrated with molecular mass reference standards mentioned above.

Activator Activity Assay In Vitro.

The results of the catalytic- and heat stable effects of the activator on PG2 , due to incubation of purified PG2 with or without the addition of purified activator are shown in FIG. 2. When pectin was incubated with purified PG2 , the amount of formed GA-units in time was determined. After heating the PG2 at 65° C. for 5 min no activity was noticeable. But, when a mixture of PG2 and activator was heated for 5 min at 65° C., pectin depolymerization was clearly perceptible. The PG2 -activator complex induced a drastic increase of pectin depolymerization. This effect was caused by complex forming of activator and PG2 , because all the remaining free PG2 was inactivated by the 65° C. incubation. So the new formed PG2 -activator complex was also heat stable at 65° C. After 30 min the substrate was the limiting factor. Incubation of activator with pectin showed no pectin-degrading activity at all.

Activator Activity In Different Plant Parts

As activator is present in other parts of the tomato plant, activator protein was isolated from leaves, roots and the stem and pectin degradation was measured. The activities of the PG2 -activator complex were calculated and expressed in nmol/h/kg tomato roots, leaves, fruits or stems. The results are summarized in Table 1.

TABLE 1
Activity of the activator in several parts of a tomato plant.

| Plant part | Activity (nmol/h/kg) |
|---|---|
| Root | 1.73 |
| Stem | 2.98 |
| Leaf | 2.02 |
| Fruit | 10.40 |

All tested parts of the tomato plant showed pectin degradation due to the formed PG2-activator complex. So, activator is present in all parts of the plant. The activity of the formed complex in fruit was 5-fold higher than the activity found in the other parts of the plant. The formed PG2 -activator complex from the roots, stem and leaves have an almost identical pectin degradation capacity.

Activator Activity In Situ

Scanning electron microscopy was used to follow the action of the activator. For that reason breaker tomato pericarp was applied because in breaker tomatoes PG2 and activator are still present. Freshly excised pericarp discs were treated with NaAc buffer or NaAc buffer containing activator. As shown in FIG. 3a and b, differences between these treatments are clearly visible. When breaker pericarp tissue is treated with NaAc (FIG. 3a), all cells and cell walls stay intact and the microfibrils are untouched. On the other hand, when pericarp tissue is treated with activator (FIG. 3b), all cell walls are demolished and even the microfibrils collapse. The lumen of the cell is completely shrivelling up and the elasticity properties of the cell disappear.

Activator Characterisation

From the UV-spectrum of the purified protein can be deduced that the protein has an absorption maximum of about 211 nm.

SDS-PAGE analysis of reduced purified protein showed a single band with a molecular mass of approximately 8.3 kDa. Determination of the molecular mass of native activator protein by gelfiltration on Sephacryl S300 yielded also a value of about 8.0 kDa.

Chromatofocusing of the purified protein indicated an iso-electric point (PI) of approximately 9.3. This result suggests that the activator is a basic protein.

A polyclonal antibody against purified β-subunit prepared by Moore is used to identify if the the β-subunit and the activator are identical. The polyclonal antibody against the β-subunit does not cross-react with the activator, only with PG1. This result indicates that the β-subunit is not analogous to the activator.

Furthermore, it was confirmed that purified activator, spotted on a membrane, stained weakly with Schiffs Reagent. This result suggest that the activator is a glycoprotein.

Purified activator shows agglutination with rabbit erythrocyte cells. This agglutination-result and other characterisation results such as, no enzymatic activity of the activator protein itself, reaction with Schiffs Reagent and binding with another glycoprotein (PG2 ) suggested that the activator is a lectin.

Protein Sequencing and Amino Acid Analysis.

Protein sequencing of gelfiltrated activator protein yielded two N-terminal sequences. The mixed protein sequence signal was as follows: (Ile/Leu)-(Ile/Ser)-(Tyr/Ala)-(Asn/Gly)-(Gln/Ala)-(Val)-(Val/Glu)-(Ser/Ala)-(Gln/Gly)-(Asp/Leu)- (Gly/Ala)-(Pro/Thr)-(Gly)-(Asp/Leu)-(Tyr/Pro)-(Gln/Tyr)-(Thr/Leu)-(Ghl/Leu)-(Ala/Gly). By means of database searches the mixed signal was split up into two signals. These two sequences are Ile-Ile-Ala-Asn-Ala-Val-Ala-Gln-Asp-Gly-Thr-Gly-Asp-Tyr-Gln-Thr-Leu-Ala (SEQ ID No. 10) and Leu-Ser-Tyr-Gly-Gln-Val-Glu-Ser-Gly-Leu-Ala-Pro-Xxx-Leu-Pro-Tyr-Leu-Gln-Gly (SEQ ID No. 11). These two sequences were respectively identified as pectinesterase and a TSW12 mRNA from tomato. Both sequences are present in equal amounts (1:1) in this gelfiltrated sample. To ensure that the N-terminal sequence of the activator matches with the amino acid sequence of the TSW 12 mRNA, the gelfiltrated sample was further purified by HPLC. The gelfiltrated sample was purified on a Nucleosil S300 column with a 120 min linear gradient from 0 to 100% acetonitril in 0.05% TFA (FIG. 4). Several peaks were observed, collected, pyridylethylated and the N-terminal amino acid sequence of the various peaks were specified. The sample that eluated from the column after 39 min contained the N-terminal amino acid sequence of the TSW 12 mRNA. From this sample an internal peptide sequence was made using the Edman degradation reaction. The amino acid sequence of this internal peptide was Ala-Ala-Gly-Ile-Pro-Ser-Ala-Xxx-Gly-Val-Ser-Ile-Pro (SEQ ID No. 12).

The amino acid composition was calculated from the gelfiltrated sample and it was found that the sample contained a mixture of two proteins (1:1) with two histidines.

TABLE 2
Calculation of amino acid composition.

| | |
|---|---|
| Comp. of sample | Data (pmol) |

| | | | |
|---|---|---|---|
| Asx | 61.6 | Asx | 308.08 |
| Glx | 49.4 | Glx | 246.98 |
| Ser | 34.7 | Ser | 173.26 |
| His | 2.0 | His | 10.12 |
| Gly | 63.7 | Gly | 318.58 |
| Thr | 38.0 | Thr | 189.80 |
| Ala | 58.0 | Ala | 289.82 |
| Arg | 22.6 | Arg | 112.83 |
| Tyr | 21.2 | Tyr | 105.88 |
| Cys | | C—C | nd |
| Val | 32.0 | Val | 159.80 |
| Met | 6.3 | Met | 31.62 |
| Phe | 17.6 | Phe | 88.23 |
| Ile | 22.2 | Ile | 110.90 |
| Leu | 42.7 | Leu | 213.46 |
| Lys | 27.7 | Lys | 138.69 |
| Pro | 20.1 | Pro | 100.36 |
| Trp | | Trp | nd |

Nucleotide Sequencing

RNA was isolated from mature green tomatos and cDNA was made using RT-PCR. Therefore, oligonucleotide primers constructed from the N-terminal amino acid sequence and the internal amino acid sequence were used. The forward primer was GGAATTCCTG(T/C)GGNCA(G/A)GTNGA(G/A)(T/A)(C/G)NGG (SEQ ID No. 13) and GGAATTCCGCN(G/C)(A/T)NGGNATNCCNGCNGC (SEQ ID No. 14) was used as the reverse primer. In both primers N indicates an inosine. After RT-PCR a cDNA fragment was isolated, ligated into a pBluescript vector, cloned in E. coli and DNA was isolated. From this DNA, the amino acid sequence (SEQ ID No. 1) comprised in the activator and the nucleotide sequence (SEQ ID No. 2) encoding SEQ ID No. 1 were determined. The nucleotide sequence was found to be 86% homologous with three nucleotide sequences, namely Lycopersicon pennellii lipid transfer protein 1 (LpLTP1) gene (SEQ ID No. 3), Lycopersicon esculentum non-specific lipid transfer protein (le16) gene (SEQ ID No. 4) and Capsicum annuum non-specific lipid transfer protein precursor gene (SEQ ID No. 16) (see FIG. 5). The amino acid sequence has a homology of 88% with the amino acid sequence of lipid transfer protein 1 from Lycopersicon esculentum (SEQ ID No. 5) and a homology of 84% with the amino acid sequence of non-specific lipid-transfer protein 2 precursor (LPT 2) from Capsicum annuum (SEQ ID No. 6) (see FIG. 6).

Computer-assisted analysis, using the Prosite-program, of the amino acid sequence of SEQ ID No. 1 indicated the presence of three N-myristoylation sites, GQVETG (SEQ ID No. 7; amino acid position 2-7 of SEQ ID No. 1), GLAPCL (SEQ ID No. 8; amino acid position 7-12 of SEQ ID No. 1) and GCCRGV (SEQ ID No. 9; amino acid position 23-28 of SEQ ID No. 1). The presence of N-myristoylation sites suggests that the protein may be covalently bound to the inner leaflet of the plasma membrane.

### FIGURES

FIG. 1 shows the isolation and purification of the activator by gelfiltration on a Sephacryl S300 column. On the X-axis the fraction number is represented and on the Y-axis the absorption at 280 nm is shown.

FIG. 2 represents activity measurements with a at 65° C. for 5 min heated combination of purified PG2 and purified activator (black dots in FIG. 2), purified PG2 (grey squares in FIG. 2), purified PG2 heated at 65° C. for 5 min (light grey triangles in FIG. 2) and purified activator heated at 65° C. for 5 min (black crosses in FIG. 2). On the X-axis the time in minutes is shown and on the Y-axis GA-units in nmol per ml are indicated.

FIG. 3 represents scanning electron micrographs of breaker tomato pericarp treated with NaAc buffer (FIG. 3a) or NaAc buffer containing activator (FIG. 3b).

FIG. 4 shows the purification of a gelfiltrated sample on a Nucleosil S300 column with a 120 min linear gradient from 0 to 100% acetonitrile in 0.05% TFA. On the X-axis the time in minutes is represented and on the Y-axis the mAU is shown.

FIG. 5 represents from top to bottom the nucleotide sequences of the activator of the present invention, Lycopersicon pennellii lipid transfer protein 1 (LpLT1), Lycopersicon esculentum non-specific lipid transfer protein (le16) and Capsicum annuum non-specific lipid transfer protein precursor, respectively.

FIG. 6 represents from top to bottom the amino acid sequence of the activator of the present invention, the amino acid sequence of lipid transfer protein 1 of Lycopersicon esculentum and the amino acid sequence of non-specific lipid-transfer protein 2 precursor (LPT 2) of Capsicum annuum, respectively.

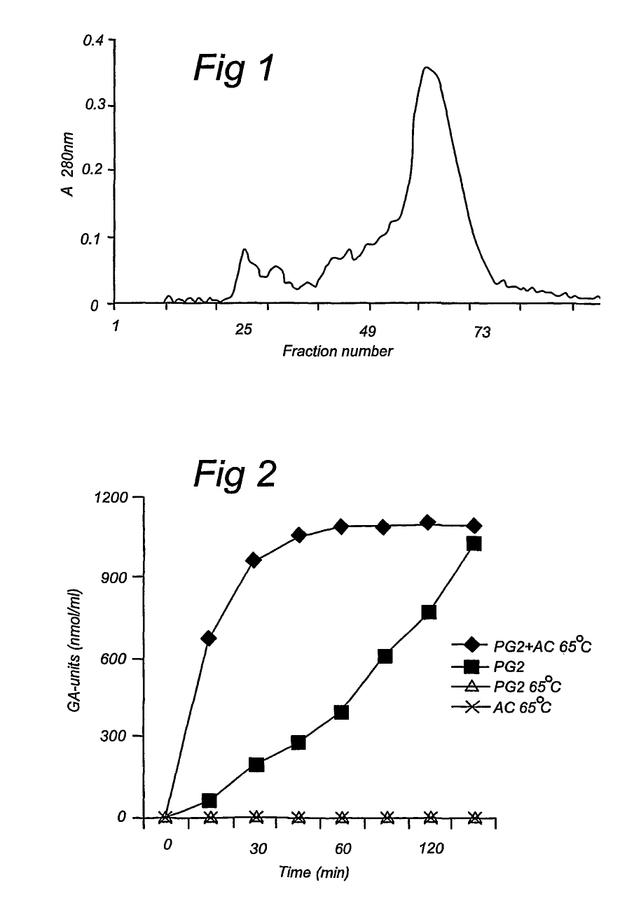

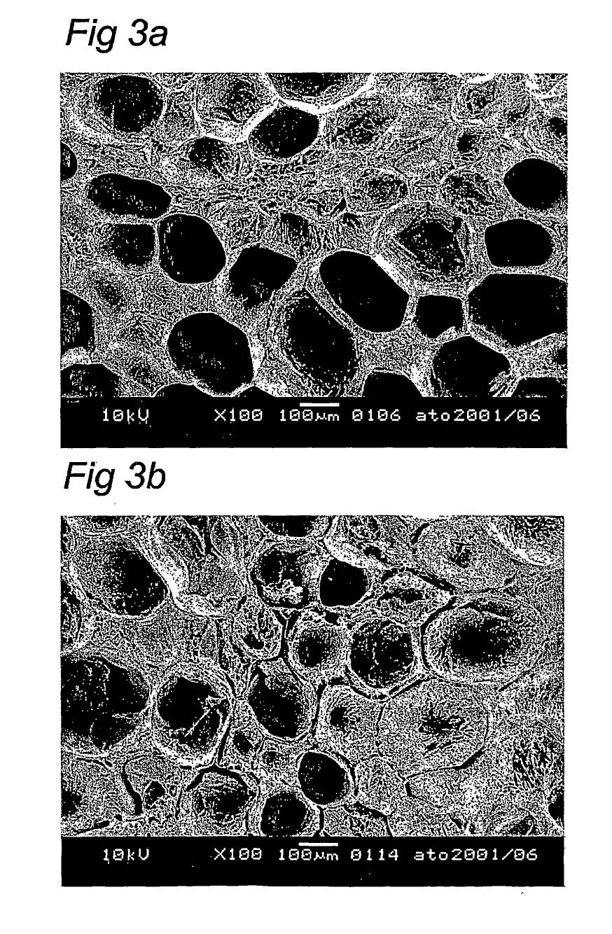

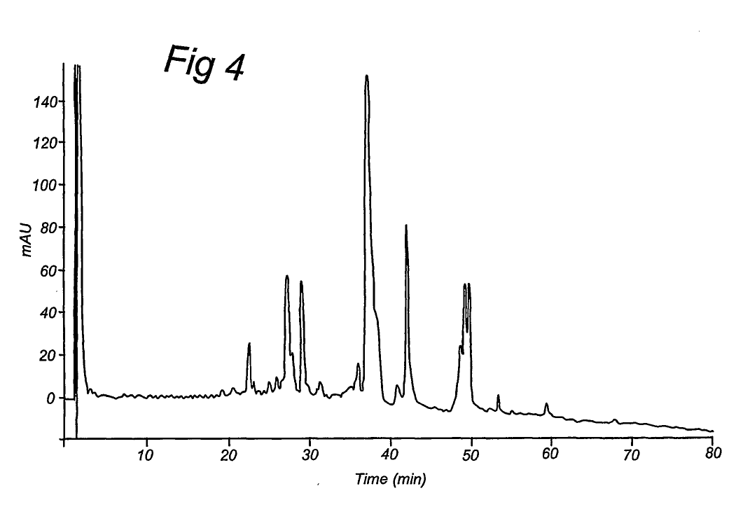

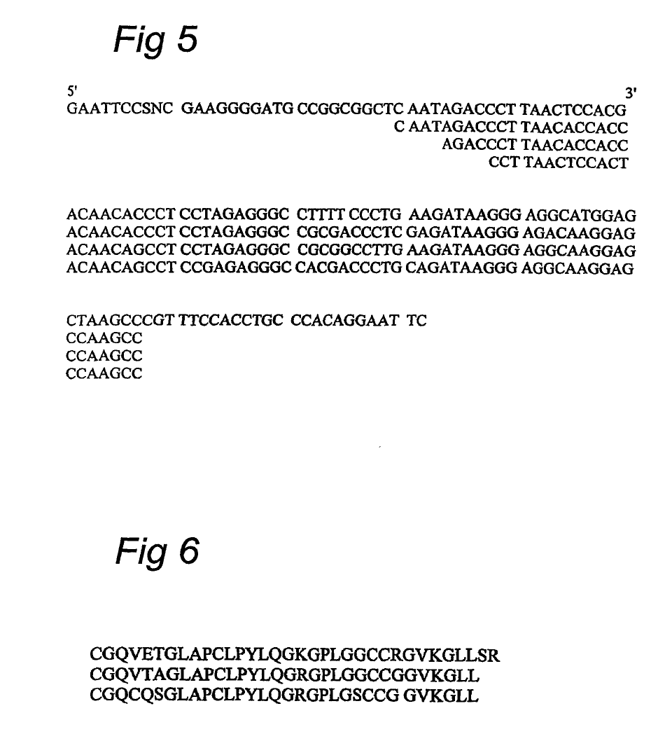

### REFERENCES

1. Chun, J. P. & Huber, D. J., (1997) Physiologia Plantarum 101, 283-290.

2. DellaPenna, D., Kates D. S. & Bennett A. B., (1987) Plant Physiol. 85, 502-507.

3. Edman, P., (1956), Acta Chem. Scand. 10, 761-768.

4. Giovannoni, J. J., DellaPenna, D., Bennett, A. B. & Fischer, R. L., (1989) The Plant Cell, Vol. 1, 53-63.

5. Harlow, E. & Lane, D., (1988) A laboratory Manual. Cold. Spring Harbor, Laboratory Press, Cold Spring Harbor, N.Y.

6. Hewick, R. M., Hunkapoller, M. W., Hood, L. E. & Dreyer, W. J., (1981) J. Siol. Chem. 15, 7990-7997.

7. Huse et al., (1989) Science 245, 1275-1281.

8. Kapitany, R. A. & Zebrowski, E. J., (1973) Anal. Biochem. 56, 361-369.

9. Knegt, E., Vermeer, E. & Bruinsma, J., (1988) Physiologia Plantarum 72, 108-114.

10. Knegt, E., Vermeer, E., Pak, C. & Bruinsma, J., (1991) Physiologia Plantarum 82, 237-242.

11. Laemmli, E. K., (1970) Nature 227, 680-685.

12. Moore, T. & Bennett, A. B., (1994) Plant Physiol. 106, 1461-1469.

13. Morrisey, J. H., (1981) Anal. Biochem. 117, 307-310.

14. Moshrefi, M. & Luh, B. S., (1983) Eur. J. Biochem. 135, 511-514.

15. Pogson, B. J., Brady, C. J. & Orr, G. R., (1991) Aust. J. Plant Physiol. 18, 65-79.

16. Pressey, R., (1984) Eur. J. Biochem. 144, 217-221.

17. Robijt, J. F., Ackerman, R. J., Keng, J. G., (1973) Anal. Biochem. 45, 517-524.

18. Rozie, H., Somers, W., Bonte, A., Visser, J., Riet van het, K. & Rombouts, F. M., (1988) Biotechnology And Applied Biochemistry 10, 346-358.

19. Schuster, R., (1988) J. Chromatog. 431, 271-284.

20. Tucker, G. A., Robertson, N. G. & Grierson, D., (1981) Eur. J. Biochem. 115, 87-90.

21. Zheng, L., Heupel, R. C. & DellaPenna, D., (1992) The Plant Cell, Vol. 4, 1147-1156.

1. A protein having the ability to activate and heat stabilise the poygalacturonase activity of purified PG2 , whereby the protein has a molecular weight of 8 kDa±2 kDa as determined by gelfiltration, and whereby the protein comprises a first amino acid sequence having at least 50% amino acid identity with the amino acid sequence of SEQ ID NO. 1.

2. A protein according to claim 1, whereby the protein further comprises a second amino acid sequence having at least 50% amino acid identity with the amino acid sequence of SEQ ID NO. 12.

3. A protein according to claim 1, whereby the protein comprises the amino acid sequence of SEQ ID NO. 15.

4. A nucleic acid molecule comprising a nucleotide sequence encoding a protein as defined in claim 1.

5. A nucleic acid molecule according to claim 4, whereby the nucleic acid molecule comprises the nucleotide sequence of SEQ ID NO. 2.

6. A nucleic acid construct comprising a nucleic acid molecule according to claim 4.

7. A nucleic acid construct according to claim 6, wherein the nucleic acid molecule is positioned so that an antisense form of the RNA of the protein is produced.

8. A transformed host comprising a nucleic acid construct according to claim 6.

9. A transformed host according to claim 8, wherein the host is selected from the group of mammalian (with the exception of human), plant, animal, fungal, yeast and bacterial cells.

10. A transformed host comprising a nucleic acid construct according to claim 7.

11. A transformed host according to claim 10, wherein the host is a plant cell.

12. A method of producing the protein according to claim 1 comprising the steps of a) culturing the transformed host under conditions conductive to the expression of the protein and b) optionally recovering the protein, or comprising the steps of a) culturing a plant naturally expressing a protein and isolating the protein, partially or completely, from the plant.

13. A composition comprising the protein according to claim 1.

14. A composition according to claim 13, wherein the composition comprises an extract of a transformed host or an extract of the culture medium thereof.

15. A composition according to claim 13, wherein the composition comprises an extract of a plant expressing a protein.

16. A method of decreasing pectin degradation by transforming a cell with a nucleic acid construct according to claim 7 or with a nucleic acid construct comprising a nucleic acid molecule encoding a protein with a mutation leading to expression of a non-functional form of said protein or leading to complete absence of expression of said protein, whereby the cell comprises lowered levels of pectin degrading proteins.

17. A transformed host according to claim 10 comprising lowered levels of a protein.

18. A method of decreasing pectin degradation by decreasing or deleting the interaction between a protein according to claim 1 and a pectin degrading protein.

19. A method of increasing pectin degradation in a preparation comprising adding a protein according to claim 1.

20. A method according to claim 19, wherein the preparation is a fruit juice.

21. A method of increasing pectin degradation in a cell comprising transforming said cell with a nucleic acid construct according to claim 6.

22. An antibody which binds specifically to the protein of claim 1 or an antigenic part thereof.

23. A cell line that produces antibodies according to claim 22.


SEQUENCE LISTING (16 sequences)

SEQ ID NO: 1 - protein, 34 aa, Lycopersicon esculentum
CGQVETGLAPCLPYLQGKGPLGGCCRGVKGLLSR

SEQ ID NO: 2 - DNA, 132 bp, Lycopersicon esculentum
  modified_base (9): a, c, g, t, other or unknown
gaattccsncgaaggggatgccggcggctcaatagacccttaactccacgacaacaccctcctagagggccttttccctgaagataagggaggcatggagctaagcccgtttccacctgcccacaggaattc

SEQ ID NO: 3 - DNA, 74 bp, Lycopersicon pennellii
agacccttaacaccaccacaacagcctcctagagggccgcggccttgaagataagggaggcaaggagccaagcc

SEQ ID NO: 4 - DNA, 78 bp, Lycopersicon esculentum
caatagacccttaacaccaccacaacaccctcctagagggccgcgaccctcgagataagggagacaaggagccaagcc

SEQ ID NO: 5 - protein, 32 aa, Lycopersicon esculentum
CGQVTAGLAPCLPYLQGRGPLGGCCGGVKGLL

SEQ ID NO: 6 - protein, 32 aa, Capsicum annuum
CGQCQSGLAPCLPYLQGRGPLGSCCGGVKGLL

SEQ ID NO: 7 - protein, 6 aa, Lycopersicon esculentum
GQVETG

SEQ ID NO: 8 - protein, 6 aa, Lycopersicon esculentum
GLAPCL

SEQ ID NO: 9 - protein, 6 aa, Lycopersicon esculentum
GCCRGV

SEQ ID NO: 10 - protein, 18 aa, Lycopersicon esculentum
IIANAVAQDGTGDYQTLA

SEQ ID NO: 11 - protein, 19 aa, Lycopersicon esculentum
  MOD_RES (13): Variable amino acid
LSYGQVESGLAPXLPYLQG

SEQ ID NO: 12 - protein, 13 aa, Lycopersicon esculentum
  MOD_RES (8): Variable amino acid
AAGIPSAXGVSIP

SEQ ID NO: 13 - DNA, 28 bp, Artificial Sequence
  Description of Artificial Sequence Synthetic primer
ggaattcctgyggncargtngarwsngg

SEQ ID NO: 14 - DNA, 28 bp, Artificial Sequence
  Description of Artificial Sequence Synthetic primer
ggaattccgcnswnggnatnccngcngc

SEQ ID NO: 15 - protein, 13 aa, Lycopersicon esculentum
LSCGQVESELAPC

SEQ ID NO: 16 - DNA, 70 bp, Capsicum annuum
ccttaactccactacaacagcctccgagagggccacgaccctgcagataagggaggcaaggagccaagcc